(19) United States
(12) Patent Application Publication
(10) Pub. No.: US 2007/0110704 A1
(43) Pub. Date: May 17, 2007

(54) Combination of 2,3-dibenzylbutyrolactone and licochalcone-a

(76) Inventors: Stefan Gallinat, Wedel (DE); Ludger Kolbe, Dohren (DE); Christopher Mummert, Bienenbuettel (DE); Rainer Wolber, Hamburg (DE); Franz Staeb, Echem (DE)

Correspondence Address: GREENBLUM & BERNSTEIN, P.L.C., 1950 ROLAND CLARKE PLACE, RESTON, VA, 20191, US

(21) Appl. No.: 10/581,271

(22) PCT Filed: Nov. 22, 2004
(86) PCT No.: PCT/EP04/13254

§ 371 Date: Aug. 31, 2006

(30) Foreign Application Priority Data
Dec. 3, 2003 (DE) 103 57 451.4

(51) Int. Cl. A61K 8/97; A61K 36/48

(52) U.S. Cl. 424/74; 424/757

(57) ABSTRACT

Active ingredient combinations of (a) one or more 2,3-dibenzylbutyrolactone derivatives and (b) licochalcone A or an aqueous extract of Radix Glycyrrhizae inflatae containing licochalcone A.

The present invention relates to cosmetic and dermatological preparations comprising active ingredients for the care and for the protection of the skin, in particular of sensitive skin, and especially of skin aged or aging by intrinsic and/or extrinsic factors, and to the use of such active ingredients and combinations of such active ingredients in the field of cosmetic and dermatological skincare.

Cosmetic skin care is primarily understood as meaning that the natural function of the skin as a barrier against environmental influences (e.g. dirt, chemicals, microorganisms) and against the loss of substances intrinsic to the body (e.g. water, natural fats, electrolytes) is strengthened or restored.

Impairment of this function may lead to increased absorption of toxic or allergenic substances or to attack by microorganisms, leading to toxic or allergic skin reactions.

In the case of aged skin, for example, regenerative renewal takes place at a slower rate, where, in particular, the water-binding capacity of the horny layer deteriorates. It therefore becomes inflexible, dry and chapped (“physiologically” dry skin). Barrier damage is the result. The skin becomes susceptible to negative environmental influences, such as the invasion of microorganisms, toxins and allergens. This may even result in toxic or allergic skin reactions.

In the case of pathologically dry and sensitive skin, barrier damage is present a priori. Epidermal intercellular lipids become defective or are formed in an inadequate amount or composition. The consequence is increased permeability of the horny layer and inadequate protection of the skin against the loss of hygroscopic substances and water.

The barrier effect of the skin can be quantified via the determination of the transepidermal water loss (TEWL). This is the evaporation of water from inside the body without taking into account the loss of water during perspiration. Determination of the TEWL value has proven to be extraordinarily informative and can be used to diagnose chapped or cracked skin, for determining the compatibility of surfactants which have very different chemical structures, and more besides.

For the beauty and well-cared-for appearance of the skin, the proportion of water in the uppermost layer of the skin is of greatest significance. It can be favorably influenced within a limited scope by introducing moisture regulators.

Anionic surfactants, which are generally constituents of cleansing preparations, can increase the pH in the horny layer with lasting effect, which severely hinders regenerative processes which serve to restore and renew the barrier function of the skin. In this case, a new, frequently very unfavorable state of equilibrium is established in the horny layer between regeneration and the loss of essential substances as a result of regular extraction; this state has a decisive adverse effect on the external appearance of the skin and the physiological mode of function of the horny layer.

Even simple bathing in water without the addition of surfactants will initially cause the horny layer of the skin to swell, the degree of this swelling depending, for example, on the bathing time and its temperature. As well as water-soluble substances, e.g. water-soluble constituents of dirt, substances which are endogenous to the skin which are responsible for the water-binding capacity of the horny layer are also washed off or out. In addition, as a result of surface-active substances endogenous to the skin, fats in the skin are also dissolved and washed out to a certain extent. After the initial swelling, this causes a subsequent significant drying-out of the skin, which may be further intensified by washing-active additives.

In healthy skin these processes are generally of no consequence since the protective mechanisms of the skin can readily compensate for such slight disturbances to the upper layers of the skin. However, even in the case of nonpathological deviations from the norm, e.g. as a result of wear damage or irritations caused by the environment, photodamage, aging skin etc., the protective mechanism of the surface of the skin is impaired. In some circumstances it is then no longer able to fulfill its role by itself and has to be regenerated by external measures.

Moreover, it is known that the lipid composition and amount of the horny layer of pathologically altered, dry and dry but not diseased skin of younger and older people deviates from the normal state found in the healthy normally hydrated skin of a group of the same age. In this connection, the changes in the lipid pattern of very dry, noneczematous skin of patients with atopic eczema represents an extreme case of the deviations which are found in the dry skin of people with healthy skin.

Here, these deviations affect very particularly the ceramides, which are severely reduced in number and additionally have a different composition. Here, the deficit of ceramides 1 and 3 is particularly striking, it being known for ceramide 1 in particular that it increases in a particular way the order of the lipids in the intercellular membrane systems.

Adverse changes in the lipid membranes of the type described above are possibly based on incorrectly controlled lipid biosynthesis and in the end effect likewise an increase in transepidermal water loss. In turn, permanent barrier weakening makes skin which is itself healthy more sensitive and can in certain instances contribute to the appearance of eczematous processes in diseased skin.

The effect of ointments and creams on barrier function and hydration of the horny layer usually does not consist in the rebuilding or strengthening of the physical-chemical properties of the lamellae of intercellular lipids. An essential partial effect is based on the mere coverage of the areas of skin treated and the blockage of water resulting therefrom in the horny layer lying below. Co-applied hygroscopic substances bind the water, resulting in a measurable increase in the water content in the horny layer. However, this purely physical barrier can be removed again relatively easily. After use of the product is stopped, the skin then reverts very quickly to the state prior to the start of treatment. Moreover, the skin care effect can decrease upon regular treatment, meaning that ultimately the status quo is again achieved even during treatment. In the case of certain products, the condition of the skin deteriorates temporarily in some circumstances when use is stopped. A permanent product effect is therefore as a rule not achieved or achieved only to a limited extent.

In order to aid deficient skin in its natural regeneration and to strengthen its physiological function, intercellular lipid mixtures have recently increasingly been added to topical preparations which are intended to be used by the skin to rebuild the natural barrier. However, these lipids, but in particular the ceramides, are very expensive raw materials. In addition, their effect is in most cases very much lower than hoped for.

The aim of the present invention was therefore to find ways to avoid the disadvantages of the prior art. In particular, the effect of skincare products should be physiological, rapid and long-lasting.

For the purposes of the present invention, skin care is understood primarily as meaning that the natural function of the skin as a barrier against environmental influences (e.g. dirt, chemicals, microorganisms) and against the loss of substances endogenous to the body (e.g. water, lipids, electrolytes) is strengthened or restored.

Products for the care, treatment and cleansing of dry and stripped skin are known per se. However, their contribution to the regeneration of a physiologically intact, hydrated and smooth horny layer is limited with regard to extent and time.

The effect of ointments and creams on the barrier function and the hydration of the horny layer is based essentially on the coverage (occlusion) of the areas of skin treated. The ointment or cream represents, as it were, a (second) artificial barrier which is intended to prevent loss of water by the skin. It is equally easy to remove this physical barrier again, for example using cleansers, as a result of which the original, impaired state is again achieved. Moreover, the skin care effect can decrease upon regular treatment. After use of the product is stopped, the skin reverts very quickly to the state prior to the start of treatment. In the case of certain products, the condition of the skin is even temporarily worsened in some circumstances. A long-lasting product effect is therefore generally not achieved or is achieved only to a limited extent.

The effect of some pharmaceutical preparations on the barrier function of the skin consists even in selective damage to the barrier, which is intended to make it possible for active ingredients to be able to penetrate into or through the skin into the body. Here, a disturbed appearance of the skin as a side-effect is accepted to some extent as a small price to pay.

The effect of caring cleansing products consists essentially in an efficient refatting with sebum lipid-like substances. The simultaneous reduction in the surfactant content of such preparations permits a further limitation of the damage to the horny layer barrier.

However, the prior art lacks preparations which have a positive influence on the barrier function and hydration of the horny layer and enhance or even restore the physicochemical properties of the horny layer and, in particular, of the lamellae comprising intercellular lipids.

The object of the present invention was therefore to overcome the disadvantages of the prior art. In particular, the aim was to provide skin care preparations and preparations for cleansing the skin which retain or restore the barrier properties of the skin, especially when the natural regeneration of the skin is inadequate. In addition, they should be suitable for the treatment and prophylaxis of damage caused by the skin drying out, for example fissures or inflammatory or allergic processes, and also neurodermitis. The object of the present invention was also to provide stable skincare cosmetic and/or dermatological compositions which protect the skin against environmental influences such as sun and wind. In particular, the effect of the preparations should be physiological, rapid and long-lasting.

Moreover, the invention relates to preparations with an extremely low so-called stinging potential. In people with sensitive, delicate or injured skin, a neurosensory phenomenon referred to as “stinging” may be observed. This “sensitive skin” differs in principle from “dry skin” with thickened and hardened horny layers.

Typical reactions of stinging in cases of sensitive skin are reddening, tightening and burning of the skin, and also itching.

Itching in cases of atopic skin is to be regarded as a neurosensory phenomenon, as is itching in cases of skin disorders.

Stinging phenomena can be regarded as being disorders to be treated cosmetically. On the other hand, severe itching, in particular severe itching arising in the case of atopy can also be referred to as a more serious dermatological disorder.

Typical troublesome neurosensory phenomena associated with the terms “stinging” or “sensitive skin” are skin reddening, tingling, prickling, tightening and burning of the skin and itching. They can be caused by stimulating environmental conditions, e.g. massage, the effect of surfactants, the influence of weather, such as sun, cold, dryness, but also moist heat, heat radiation and UV radiation, e.g. of the sun.

In “Journal of the Society of Cosmetic Chemists” 28, pp. 197-209 (May 1977), P. J. Frosch and A. M. Kligman describe a method for estimating the stinging potential of topically administered substances. The positive substances used here are, for example, lactic acid and pyruvic acid. When measuring in accordance with this method, however, amino acids, in particular glycine, were also found to be neurosensorially active (such substances are called “stingers”).

According to findings to date, such sensitivity to very specific substances varies from person to person. This means that someone who experiences stinging effects upon contact with a substance will experience them again with high probability upon each further contact. Contact with other “stingers” may, however, take place just as well without any reaction.

The problem of sensitive skin affects a growing number of adults and children. Sensitive skin is used to refer to a combination of various symptoms, such as hyperreactive and intolerant skin. However, atopic skin can also be subsumed under this. These skin conditions are often, not entirely correctly, referred to by those affected as “allergic” skin. Although an allergic disorder can lead to symptoms of sensitive skin, the appearance of sensitive skin is not restricted to those with allergies.

Many individuals of greater or lesser sensitivity also have to suffer from erythematous skin symptoms when using some deodorizing or antiperspirant preparations.

Erythematous skin symptoms also arise as accompanying symptoms of certain skin disorders or irregularities. For example, the typical skin rash in the case of the appearance of acne is often red to a greater or lesser degree.

It was therefore the object of the present invention to overcome the disadvantages of the prior art.

In particular, active ingredients and preparations comprising such active ingredients were to be made available for the cosmetic and dermatological treatment and/or prophylaxis of erythematous, inflammatory, allergic or autoimmune-reactive symptoms, in particular dermatoses, but also of the development of “stinging”.

In addition, such active ingredients, or preparations comprising such active ingredients were to be made available which can be used for the immunostimulation of the skin, here advantageously for immunostimulation in the sense of the effect promoting wound healing.

In a further preferred embodiment, the present invention relates to cosmetic and dermatological preparations for the prophylaxis and treatment of cosmetic or dermatological changes in the skin, such as, for example, undesired pigmentation, for example local hyperpigmentation and incorrect pigmentation (for example liver spots, freckles), or for the purely cosmetic lightening of larger areas of skin which are quite appropriately pigmented for the individual skin type.

Pigmenting of the skin is caused, for example, by melanocytes, which are to be found in the lowest layer of the epidermis, the Stratum basale, alongside the basal cells as pigment-forming cells which, depending on the skin type, occur either individually or in clusters of varying size. Melanocytes contain, as characteristic cell organelles, melanosomes which form melanin to a greater extent when stimulated by UV radiation. This melanin is transported into the keratinocytes and brings about a more or less marked brownish or brown skin color.

Melanin is formed as the end stage of an oxidation process in which tyrosine is finally converted into melanin, under the action of the enzyme tyrosinase, via 3,4-dihydroxyphenylalanine (dopa), dopaquinone, leucodopachrome, dopachrome, 5,6-dihydroxyindole and indole-5,6-quinone.

Problems with skin hyperpigmentation have many different causes and are accompanying phenomena of many biological processes, for example UV radiation (for example freckles, Ephelides), genetic disposition, incorrect pigmentation of the skin during wound healing or scarring or skin aging (for example Lentigines seniles).

Active ingredients and preparations which counteract skin pigmentation are known. In practice, use is made essentially of preparations based on hydroquinone although, on the one hand, these only show their effect after application for several weeks and, on the other hand, application of them for an excessively long time is not always without risk, for toxicological reasons. The inhibition of tyrosinase with substances such as kojic acid, ascorbic acid and azelaic acid and their derivatives is also common, although it has cosmetic and dermatological disadvantages.

The object of the present invention was also to remedy these shortcomings.

Another aim of skin care is to compensate for the loss by the skin of lipids and water caused by daily washing. This is particularly important when the natural regeneration ability is inadequate. Furthermore, skin care products should protect against environmental influences, in particular against sun and wind, and delay skin aging.

Chronological skin aging is caused, for example, by endogenous, genetically determined factors. The following structural damage and functional disorders, which can also fall under the term “senile xerosis”, arise, for example, in the epidermis and dermis as a result of aging:

a) dryness, roughness and formation of dryness wrinkles,

b) itching and

c) reduced refatting by sebaceous glands (e.g. after washing).

Exogenous factors, such as UV light and chemical noxae, can have a cumulative effect and, for example, accelerate or supplement the endogenous aging processes. In the epidermis and dermis, for example, the following structural damage and functional disorders arise in the skin in particular as a result of exogenous factors; these are more far-reaching than the degree and quality of the damage in the case of chronological aging:

d) visible vascular dilation (telangiectases, couperosis);

e) flaccidity and formation of wrinkles;

f) local hyperpigmentation, hypopigmentation and abnormal pigmentation (e.g. age spots) and

g) increased susceptibility to mechanical stress (e.g. chapping).

The present invention relates in particular to products for the care of skin aged naturally, and to the treatment of the damage caused by photoaging, in particular of the phenomena listed under a) to g).

Products for the care of aged skin are known per se. They comprise, for example, retinoids (vitamin A acid and/or derivatives thereof) or vitamin A and/or derivatives thereof. Their effect on structural damage is, however, limited. Furthermore, in product development there are considerable difficulties in stabilizing the active ingredients to an adequate extent against oxidative decay. The use of products comprising vitamin A acid, moreover, often causes severe erythematous skin irritations. Retinoids can therefore only be used in low concentrations.

In particular, the present invention relates to cosmetic preparations having effective protection against harmful oxidation processes in the skin, but also for the protection of cosmetic preparations themselves or for the protection of the constituents of cosmetic preparations against harmful oxidation processes.

The present invention further relates to antioxidants, preferably those used in skin care cosmetic or dermatological preparations. In particular, the invention also relates to cosmetic and dermatological preparations comprising such antioxidants. In a preferred embodiment, the present invention relates to cosmetic and dermatological preparations for the prophylaxis and treatment of cosmetic or dermatological skin changes, such as, for example, skin aging, in particular skin aging caused by oxidative processes.

Furthermore, the present invention relates to active ingredients and preparations comprising such active ingredients for the cosmetic and dermatological treatment or prophylaxis of erythematous, inflammatory, allergic or autoimmune-reactive symptoms, in particular dermatoses.

In a further advantageous embodiment, the present invention relates to active ingredient combinations and preparations which serve for the prophylaxis and treatment of light-sensitive skin, in particular of photodermatoses.

The harmful effect of the ultraviolet part of solar radiation on the skin is generally known. Whereas rays with a wavelength of less than 290 nm (the UVC region) are absorbed by the ozone layer in the earth's atmosphere, rays in the range between 290 nm and 320 nm, the UVB region, cause erythema, simple sunburn or even burns of greater or lesser severity.

A maximum erythema activity of sunlight is given as the relatively narrow range around 308 nm.

Numerous compounds are known for protecting against UVB radiation; these are derivatives of 3-benzylidenecamphor, of 4-aminobenzoic acid, of cinnamic acid, of salicylic acid, of benzophenone and also of 2-phenylbenzimidazole.

It is also important to have available filter substances for the range between about 320 nm and about 400 nm, the UVA region, since its rays can cause reactions in cases of photosensitive skin. It has been found that UVA radiation leads to damage of the elastic and collagenous fibers of connective tissue, which leads to premature aging of the skin, and is to be regarded as a cause of numerous phototoxic and photoallergic reactions. The harmful effect of UVB radiation can be intensified by UVA radiation.

To protect against rays of the UVA region, therefore, certain derivatives of dibenzoylmethane are used, the photostability of which is inadequate (Int. J. Cosm. Science 10, 53 (1988)).

The UV radiation can, however, also lead to photochemical reactions, in which case the photochemical reaction products then intervene in the skin's metabolism.

Such photochemical reaction products are predominantly free-radical compounds, for example hydroxyl radicals, singlet oxygen. Undefined free-radical photoproducts which form in the skin itself can also display uncontrolled secondary reactions because of their high reactivity. However, singlet oxygen, a non-free-radical excited state of the oxygen molecule, can also be formed during UV irradiation, as can short-lived epoxides and many others. Singlet oxygen, for example, differs from normal triplet oxygen (free-radical ground state) by virtue of its increased reactivity. However, excited, reactive (free-radical) triplet states of the oxygen molecule also exist.

UV radiation is also a type of ionizing radiation. There is therefore the risk that ionic species will also form during UV exposure, which then for their part are able to intervene oxidatively in the biochemical processes.

In order to prevent these reactions, additional antioxidants and/or free-radical scavengers can be incorporated into the cosmetic or dermatological formulations.

It has already been proposed to use vitamin E, a substance with known antioxidative action, in light protection formulations, although, here too, the effect achieved falls a long way short of expectations.

The object of the invention was therefore to provide cosmetic, dermatological and pharmaceutical active ingredients and preparations, and light protection formulations which serve for the prophylaxis and treatment of photosensitive skin, in particular photodermatoses, preferably PLD.

Other names for polymorphous photodermatosis are PLD, PLE, Mallorca acne and a large number of other names, as given in the literature (e.g. A. Voelckel et al, Zentralblatt Haut-und Geschlechtskrankheiten (1989), 156, p. 2).

Antioxidants are mainly used as substances which protect against the deterioration of the preparations in which they are present. Nevertheless, it is known that in human or animal skin as well, undesired oxidation processes may occur. Such processes play an important role in skin aging.

The essay “Skin Diseases Associated with Oxidative Injury” in “Oxidative Stress in Dermatology”, p. 323 ff. (Marcel Decker Inc., New York, Basel, Hong Kong, Editor: Jürgen Fuchs, Frankfurt, and Lester Packer, Berkeley/Calif.) discusses oxidative skin damage and its more obvious causes.

Also for the reason of preventing such reactions, antioxidants and/or free-radical scavengers can be additionally incorporated into cosmetic or dermatological formulations.

A number of antioxidants and free-radical scavengers are known. For example U.S. Pat. Nos. 4,144,325 and 4,248,861, and numerous other documents have already proposed the use of vitamin E, a substance with known antioxidative action in light protection formulations, although here too the effect achieved falls a long way short of the desired effect.

An object of the present invention was therefore to find ways to avoid the disadvantages of the prior art. In particular, the effect of eliminating the damage associated with endogenous, chronological and exogenous skin aging and the prophylaxis should be permanent, long-lasting and without the risk of secondary effects.

According to the invention, the shortcomings of the prior art are eliminated by active ingredient combinations of
(a) one or more 2,3-dibenzylbutyrolactone derivatives and
(b) licochalcone A or an aqueous extract of Radix Glycyrrhizae inflatae, containing licochalcone A
   or cosmetic or dermatological preparations comprising such active ingredient combinations.

Preparations according to the invention or cosmetic or dermatological preparations are entirely satisfactory preparations in every respect. It could not have been foreseen by the person skilled in the art that the preparations according to the invention
better maintain or restore the barrier properties of the skin,
better counteract the skin drying out,
better act against pigment disorders,
better act against inflammatory skin conditions
better act against skin ageing and
better protect the skin against environmental influences
   than the preparations of the prior art.

The use of active ingredient combinations according to the invention or cosmetic or topical dermatological preparations with an effective content of active ingredient combinations according to the invention surprisingly offers effective treatment, but also prophylaxis
of deficient, sensitive or hypoactive skin conditions or deficient, sensitive or hypoactive states of skin appendages,
of symptoms of premature aging of the skin (e.g. wrinkles, age spots, telangiectases) and/or of the skin appendages,
of environmentally induced changes in the skin and the skin appendages (smoking, smog, reactive oxygen species, free radicals) and in particular light-induced negative changes,
of light-induced skin damage,
of pigmentation disorders,
of sensitive, irritated and itching skin,
of dry skin conditions and impairment of the horny layer barrier,
of hair loss and for improved hair growth,
of inflammatory skin conditions such as atopic eczema, seborrhoeic eczema, polymorphous photodermatosis, psoriasis, vitiligo.

The active ingredient according to the invention or cosmetic or topical dermatological preparations with an effective content of active ingredient according to the invention, however, also surprisingly serves
to calm sensitive or irritated skin,
to stimulate the synthesis of collagen, hyaluronic acid and elastin,
to stimulate the synthesis of ceramide in the skin,
to stimulate intracellular DNA synthesis, in particular in cases of deficient or hypoactive skin conditions,
to increase cell renewal and regeneration of the skin,
to increase the skin's own protective and repair mechanisms (for example for dysfunctional enzymes, DNA, lipids, proteins),
for the pre- and post-treatment in cases of topical application of laser and abrasive treatments, which serve, for example, to reduce skin wrinkles and scars, to counteract the resulting skin irritations and to promote the regeneration processes in the damaged skin.

Preferably, in the active ingredient combinations according to the invention, the 2,3-dibenzylbutyrolactone derivative or derivatives and licochalcone A are present in ratios of from 50:1 to 1:50, preferably 10:1 to 1:10, particularly preferably 2:1 to 1:2.

The 2,3-dibenzylbutyrolactone derivatives according to the invention and/or glycosides thereof are derived from 2,3-dibenzylbutyrolactone, which is likewise in accordance with the invention and which is characterized by the following structure:

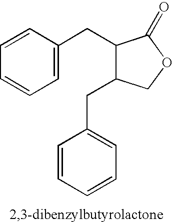

2,3-Dibenzylbutyrolactone, 2,3-dibenzylbutyrolactone derivatives and/or glycosides thereof in all of their stereoisomeric forms, which may be present either as racemate or in enantiomerically pure form, and also in racemic mixtures with varying enantiomer fractions, are in accordance with the invention. According to the invention, the formulation 2,3-dibenzylbutyrolactone derivatives and/or glycosides thereof also includes 2,3-dibenzylbutyrolactone.

The 2,3-dibenzylbutyrolactone derivatives according to the invention and/or glycosides thereof can be added to the preparation according to the invention advantageously in the form of plant extracts. In this connection, aqueous-alcoholic extracts from plants have proven particularly useful. However, extracts and distillates obtained using other extraction forms and methods, for example extracts and steam distillates obtained using carbon dioxide as extractant, are also to be formulated advantageously according to the invention into the preparations.

In this connection, it is particularly advantageous according to the invention to use plant extracts of Arctium lappa L. (great burdock) and/or Steganotaenia araliacea (carrot tree).

2,3-Dibenzylbutyrolactone derivatives preferred according to the invention and/or glycosides thereof used are arctiin, arctigenin, prestegan B, matairesinol, tracheloside and/or trachelogenin.

In this connection, particular preference is given according to the invention to the derivatives with the following stereochemical structure:

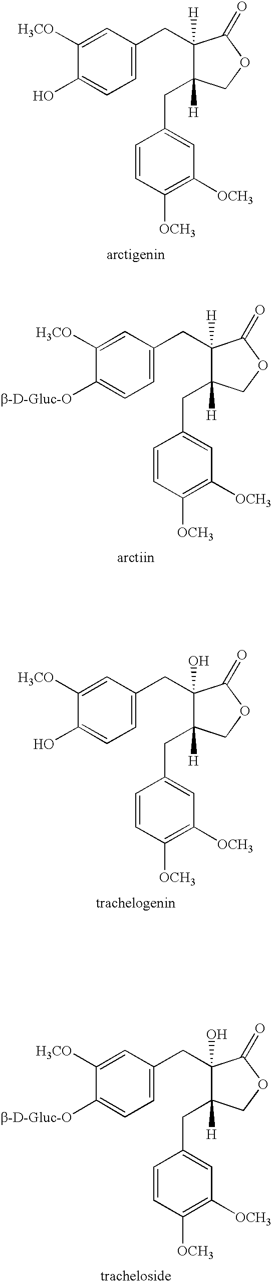

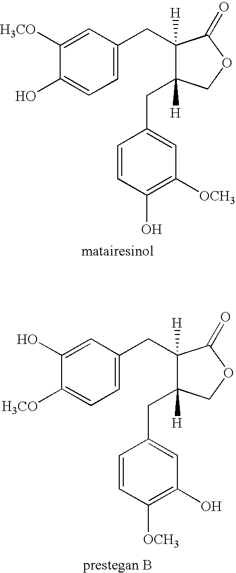

According to the invention, very particular preference is given to arctiin and prestegan B.

The 2,3-dibenzylbutyrolactone derivatives according to the invention and/or glycosides thereof can be incorporated without problems into customary cosmetic and/or dermatological preparations, such as sunscreen preparations, skin care preparations, antiwrinkle preparations, but also other preparations, for example pharmaceutical preparations.

Advantageously, preparations according to the invention comprise 0.01-25% by weight of one or more 2,3-dibenzylbutyrolactone derivatives, preferably 0.1-20% by weight, in particular 1-10% by weight, in each case based on the total weight of the preparations.

Like the licorice Glycyrrhiza glabra officinal in Europe, the plant species Glycyrrhiza inflata belongs to the genus Glycyrrhiza, which belongs to the Fabaceae plant family (pea plants). The drug Radix Glycyrrhizae inflatae, i.e. the root of the plant, is used widely, for example, in Far Eastern medicine. Use of the drug as anti-inflammatory is likewise known.

One constituent of the aqueous extract of Radix Glycyrrhizae inflatae is licochalcone A which is characterized by the following structural formula:

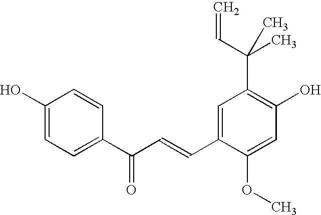

It is assumed that this substance, possibly in synergy with the other constituents of the extract, has part of the effect according to the invention.

According to the invention, it is particularly advantageous if the preparations comprise 0.0001 to 5% by weight, in particular 0.001 to 1% by weight, very particularly 0.005 to 0.15% by weight, of licochalcone A, based on the total weight of the preparation.

According to the invention, it is also advantageous in particular if the preparations comprise 0.001 to 10% by weight, in particular 0.05 to 5% by weight, very particularly 0.01 to 2% by weight, of one or more polyols, based on the total weight of the preparation.

According to the invention, it is also advantageous if the preparations comprise licochalcone as constituent of plant extracts, in particular of Radix Glycyrrhizae inflatae.

According to the invention, it is also advantageous if licochalcone is present in the form of an aqueous extract in which
licochalcone A
water
optionally one or more polyols
   are present.

According to the invention, it is advantageous if the cosmetic or dermatological preparations comprise 0.001 to 10% by weight, in particular 0.05 to 5% by weight, very particularly 0.01 to 2% by weight, of an aqueous extract from Radix Glycyrrhizae inflatae, based on the total weight of the preparation.

It is advantageous according to the invention if the cosmetic or dermatological preparations comprise 0.001 to 10% by weight, in particular 0.05 to 5% by weight, very particularly 0.01 to 2% by weight, of one or more polyols, based on the total weight of the preparation.

In particular, it is advantageous to choose butylene glycol as polyol.

It is very particularly advantageous to start from an extract which is sold under the name Polyol Soluble Licorice Extract P-U by Maruzen.

It is also advantageous to use licochalcone A in other vehicle systems in a concentration of 0.0001 to 5% by weight, in particular 0.001 to 1% by weight, very particularly 0.005-0.05% by weight.

Accordingly, the use of active ingredient combinations according to the invention for the prophylaxis and treatment of inflammatory skin conditions—including atopic eczema—and/or for protecting the skin in cases of sensitively determined dry skin is in accordance with the invention.

Accordingly, the use of active ingredient combinations according to the invention for producing cosmetic or dermatological preparations for producing cosmetic or dermatological preparations for the treatment and/or prophylaxis of pigment disorders is in accordance with the invention.

Accordingly, the use of active ingredient combinations according to the invention for producing cosmetic or dermatological preparations for the treatment and/or prophylaxis of the symptoms of intrinsic and/or extrinsic skin aging, and for the treatment and prophylaxis of the harmful effects of ultraviolet radiation on the skin is in accordance with the invention.

Accordingly, the use of active ingredient combinations according to the invention for producing cosmetic or dermatological preparations for increasing ceramide biosynthesis is also in accordance with the invention.

Accordingly, the use of active ingredient combinations according to the invention for producing cosmetic or dermatological preparations for enhancing the barrier function of the skin is also in accordance with the invention.

Preferably, cosmetic or dermatological preparations according to the invention comprise 0.001-10% by weight, particularly preferably 0.01-1% by weight, of active ingredient combinations according to the invention, based on the total composition of the preparations.

In particular, it is extremely advantageous according to the invention to use active ingredient combinations according to the invention or cosmetic or topical dermatological preparations with an effective content of active ingredient combinations according to the invention for the cosmetic or dermatological treatment or prophylaxis of undesired skin conditions.

According to the invention, customary antioxidants may be used in preparations which comprise active ingredient combinations according to the invention.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoides, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate) tocopherols and derivatives (e.g. vitamin E acetate), vitamin A and derivatives (vitamin A palmitate) and coniferyl benzoate of benzoin resin, rutinic acid and derivatives thereof, α-glycosylrubin ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of these said active ingredients which are suitable according to the invention.

The amount of antioxidants (one or more compounds) in the preparations is preferably 0.001 to 30% by weight, particularly preferably 0.05-20% by weight, in particular 1-10% by weight, based on the total weight of the preparation.

The prophylaxis or the cosmetic or dermatological treatment with the active ingredient used according to the invention or with the cosmetic or topical dermatological preparations with an active content of active ingredient used according to the invention is carried out in the usual manner, by applying the active ingredient used according to the invention or the cosmetic or topical dermatological preparations with an active content of active ingredient used according to the invention to the affected areas of skin.

The active ingredient used according to the invention can advantageously be incorporated into customary cosmetic and dermatological preparations, which may be in various forms. Thus, they may, for example, be a solution, an emulsion of the water-in-oil (W/O) type or of the oil-in-water (O/W) type, or a multiple emulsion, for example of the water-in-oil-in-water (W/O/W) type or oil-in-water-in-oil (O/W/O) type, a hydrodispersion or lipodispersion, a gel, a solid stick or an aerosol.

Emulsions according to the invention for the purposes of the present invention, e.g. in the form of a cream, a lotion, a cosmetic milk, are advantageous and comprise, for example, fats, oils, waxes and/or other fatty substances, and water and one or more emulsifiers as are customarily used for this type of formulation.

It is also possible and advantageous for the purposes of the present invention to incorporate the active ingredient used according to the invention into aqueous systems or surfactant preparations for cleansing the skin and the hair.

The person skilled in the art is of course aware that demanding cosmetic compositions are mostly inconceivable without the customary auxiliaries and additives. The cosmetic preparations according to the invention can therefore comprise cosmetic auxiliaries, as are customarily used in such preparations, e.g. preservatives, bactericides, deodorizing substances, antiperspirants, insect repellents, vitamins, antifoams, dyes, pigments with a coloring action, thickeners, softening substances, moisturizing substances and/or humectant substances, fats, oils, waxes or other customary constituents of a cosmetic formulation, such as alcohols, polyols, polymers, foam stabilizers, electrolytes, organic solvents or silicone derivatives.

Corresponding requirements apply mutatis mutandis to the formulation of medicinal preparations.

Medicinal topical compositions for the purposes of the present invention generally comprise one or more medicaments in an effective concentration. For the sake of simplicity, for a clear distinction between cosmetic and medicinal application and corresponding products, reference is made to the legal provisions of the Federal Republic of Germany (e.g. Cosmetics Directive, Foods and Drugs Act).

Furthermore, preparations according to the invention can advantageously comprise substances which absorb UV radiation in the UVB region, where the total amount of the filter substances is, for example, 0.1% by weight to 30% by weight, preferably 0.5 to 10% by weight, in particular 1.0 to 6.0% by weight, based on the total weight of the preparations, in order to provide cosmetic preparations which protect the hair and/or the skin from the entire range of ultraviolet radiation. They can also be used as sunscreens for the hair.

If the preparations according to the invention comprise UVB filter substances, these may be oil-soluble or water-soluble. Examples of oil-soluble UVB filters which are advantageous according to the invention are:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethylamino)benzoate, amyl 4-(dimethylamino)benzoate;
esters of cinnamic acid, preferably 2-ethylhexyl 4-methoxycinnamate, isopentyl 4-methoxycinnamate;
esters of salicylic acid, preferably 2-ethylhexyl salicylate, 4-isopropylbenzyl salicylate, homomenthyl salicylate,
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di(2-ethylhexyl)4-methoxybenzalmalonate,
2,4,6-trianilino(p-carbo-2′-ethyl-1′-hexyloxy)-1,3,5-triazine.

Advantageous water-soluble UVB filters are, for example:
salts of 2-phenylbenzimidazole-5-sulfonic acid, and its sodium, potassium or its triethanolammonium salt, and the sulfonic acid itself;
sulfonic acid derivatives of benzophenones, preferably 2-hydroxy-4-methoxybenzo-phenone-5-sulfonic acid and its salts;
sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and its salts, and also 1,4-di(2-oxo-10-sulfo-3-bornylidenemethyl)benzene and salts thereof (the corresponding 10-sulfato compounds, for example the corresponding sodium, potassium or triethanolammonium salt), also referred to as benzene-1,4-di(2-oxo-3-bornylidenemethyl-10-sulfonic acid.

The list of specified UVB filters which can be used in combination with the active ingredient combinations according to the invention is not of course intended to be limiting.

It may also be advantageous to use UVA filters which are customarily present in cosmetic preparations. These substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. The amounts used for the UVB combination may be used.

Furthermore, cosmetic and dermatological preparations according to the invention advantageously comprise inorganic pigments based on metal oxides and/or other metal compounds which are sparingly soluble or insoluble in water, in particular the oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides. They are particularly preferably pigments based on TiO₂.

For the purposes of the present invention, it is particularly advantageous, although not obligatory, for the inorganic pigments to be in hydrophobic form, i.e. to have been treated superficially to repel water. This surface treatment can consist in providing the pigments with a thin hydrophobic layer by methods known per se.

Such a method consists, for example, in producing the hydrophobic surface layer by a reaction according to
nTiO₂+m(RO)₃ Si—R′->nTiO₂ (surf.)
n and m here are stoichiometric parameters to be used as desired, R and R′ are the desired organic radicals. Hydrophobicized pigments synthesized, for example, in analogy to DE-A 33 14 742 are advantageous.

Advantageous TiO₂ pigments are available, for example, under the trade names MT 100 T from TAYCA, also M 160 from Kemira, and T 805 from Degussa.

Preparations according to the invention can, especially if crystalline or microcrystalline solids, for example inorganic micropigments, are to be incorporated into the preparations according to the invention, also comprise anionic, nonionic and/or amphoteric surfactants. Surfactants are amphiphilic substances which can dissolve organic, nonpolar substances in water.

The hydrophilic moieties of a surfactant molecule are mostly polar functional groups, for example —COO⁻, —OSO₃²⁻, —SO₃⁻, whereas the hydrophobic moieties are usually nonpolar hydrocarbon radicals. Surfactants are generally classified according to the type and charge of the hydrophilic molecular moiety. In this connection, it is possible to differentiate between four groups:
anionic surfactants,
cationic surfactants,
amphoteric surfactants and
nonionic surfactants.

Anionic surfactants usually have, as functional groups, carboxylate, sulfate or sulfonate groups. In aqueous solution, they form negatively charged organic ions in an acidic or neutral medium. Cationic surfactants are characterized almost exclusively by the presence of a quaternary ammonium group. In aqueous solution, they form positively charged organic ions in an acidic or neutral medium. Amphoteric surfactants contain both anionic and cationic groups and accordingly in aqueous solution exhibit the behavior of anionic or cationic surfactants depending on the pH. In a strongly acidic medium, they have a positive charge, and in an alkali medium a negative charge. By contrast, in the neutral pH range, they are zwitterionic, as the example below is intended to illustrate:

| | | |
|---|---|---|
| RNH₂⁺CH₂CH₂COOH X⁻ | (at pH = 2) | X⁻ = any anion, e.g. Cl⁻ |
| RNH₂⁺CH₂CH₂COO⁻ | (at pH = 7) | |
| RNHCH₂CH₂COO⁻ B⁺ | (at pH = 12) | B⁺ = any cation, e.g. Na⁺ |

Typical nonionic surfactants are polyether chains. Nonionic surfactants do not form ions in aqueous medium.

A. Anionic Surfactants.

Anionic surfactants which can be used advantageously are acylamino acids (and salts thereof), such as
1. acyl glutamates, for example sodium acyl glutamate, di-TEA-palmitoyl aspartate and sodium caprylic/capric glutamate,
2. acylpeptides, for example palmitoyl-hydrolyzed milk protein, sodium cocoyl-hydrolyzed soya protein and sodium/potassium cocoyl-hydrolyzed collagen,
3. sarcosinates, for example myristoyl sarcosine, TEA-lauroyl sarcosinate, sodium lauroyl sarcosinate and sodium cocoyl sarcosinate,
4. taurates, for example sodium lauroyl taurate and sodium methyl cocoyl taurate,
5. acyl lactylates, lauroyl lactylate, caproyl lactylate
6. alaninates
   carboxylic acids and derivatives, such as
1. carboxylic acids, for example lauric acid, aluminum stearate, magnesium alkanolate and zinc undecylenate,
2. ester carboxylic acids, for example calcium stearoyl lactylate, laureth-6 citrate and sodium PEG4 lauramide carboxylate,
3. ether carboxylic acids, for example sodium laureth-13 carboxylate and sodium PEG-6 cocamide carboxylate,
   phosphoric esters and salts, such as, for example, DEA-oleth-10 phosphate and dilaureth-4 phosphate,
   sulfonic acids and salts, such as
1. acyl isethionates, e.g. sodium/ammonium cocoyl isethionate,
2. alkylarylsulfonates,
3. alkylsulfonates, for example sodium cocomonoglyceride sulfate, sodium C₁₂₋₁₄-olefinsulfonate, sodium lauryl sulfoacetate and magnesium PEG-3 cocamide sulfate,
4. sulfosuccinates, for example dioctyl sodium sulfosuccinate, disodium laureth sulfosuccinate, disodium lauryl sulfosuccinate and disodium undecyleneamido-MEA sulfosuccinate
   and
   sulfuric esters, such as
1. alkyl ether sulfate, for example sodium, ammonium, magnesium, MIPA, TIPA laureth sulfate, sodium myreth sulfate and sodium C₁₂₋₁₃-parethsulfate,
2. alkyl sulfates, for example sodium, ammonium and TEA lauryl sulfate.
   B. Cationic Surfactants

Cationic surfactants which can be used advantageously are
1. alkylamines,
2. alkylimidazoles,
3. ethoxylated amines and
4. quaternary surfactants.
5. ester quats

Quaternary surfactants comprise at least one N atom which is covalently bonded to 4 alkyl and/or aryl groups. Irrespective of the pH, this leads to a positive charge. Alkylbetaine, alkylamidopropylbetaine and alkylamidopropylhydroxysulfaine are advantageous. The cationic surfactants used according to the invention can also be preferably chosen from the group of quaternary ammonium compounds, in particular benzyltrialkylammonium chlorides or bromides, such as, for example, benzyldimethylstearylammonium chloride, and also alkyltrialkylammonium salts, for example cetyltrimethylammonium chloride or bromide, alkyldimethylhydroxyethyl-ammonium chlorides or bromides, dialkyldimethylammonium chlorides or bromides, alkylamidoethyltrimethylammonium ether sulfates, alkylpyridinium salts, for example lauryl- or cetylpyrimidinium chloride, imidazoline derivatives and compounds with a cationic character, such as amine oxides, for example alkyl dimethylamine oxides or alkylaminoethyldimethylamine oxides. In particular, the use of cetyltrimethylammonium salts is advantageous.

C. Amphoteric Surfactants

Amphoteric surfactants which can be used advantageously are
1. acyl/dialkylethylenediamine, for example sodium acyl amphoacetate, disodium acyl amphodipropionate, disodium alkyl amphodiacetate, sodium acyl amphohydroxypropylsulfonate, disodium acyl amphodiacetate and sodium acyl amphopropionate,
2. N-alkylamino acids, for example aminopropylalkylglutamide, alkylaminopropionic acid, sodium alkylimidodipropionate and lauroamphocarboxyglycinate.
   D. Nonionic Surfactants

Nonionic surfactants which can be used advantageously are
1. alcohols,
2. alkanolamides, such as cocamides MEA/DEA/MIPA,
3. amine oxides, such as cocoamidopropylamine oxide,
4. esters which are formed by esterification of carboxylic acids with ethylene oxide, glycerol, sorbitan or other alcohols,
5. ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glycoside and cocoglycoside
6. sucrose esters, sucrose ethers
7. polyglycerol esters, diglycerol esters, monoglycerol esters
8. methyl glucose esters, esters of hydroxy acids.

Also advantageous is the use of a combination of anionic and/or amphoteric surfactants with one or more nonionic surfactants.

The surface-active substance may be present in the preparations according to the invention in a concentration between 1 and 95% by weight, based on the total weight of the preparations.

The lipid phase of the cosmetic or dermatological emulsions according to the invention can advantageously be chosen from the following group of substances:
mineral oils, mineral waxes
oils, such as triglycerides of capric or of caprylic acid, and also natural oils such as, for example, castor oil;
fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;
alkyl benzoates;
silicone oils, such as dimethylpolysiloxanes, diethylpolysiloxanes, diphenylpolysiloxanes and mixed forms thereof.

The oil phase of the emulsions of the present invention is advantageously chosen from the group of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, from the group of esters of aromatic carboxylic acids and saturated and/or unsaturated, branched and/or unbranched alcohols having a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

In addition, the oil phase can advantageously be chosen from the group of branched and unbranched hydrocarbons and hydrocarbon waxes, of silicone oils, of dialkyl ethers, the group of saturated or unsaturated, branched or unbranched alcohols, and the fatty acid triglycerides, namely the triglycerol esters of saturated and/or unsaturated, branched and/or unbranched alkanecarboxylic acids having a chain length of from 8 to 24, in particular 12-18 carbon atoms. The fatty acid triglycerides can, for example, advantageously be chosen from the group of synthetic, semisynthetic and natural oils, e.g. olive oil, sunflower oil, soybean oil, groundnut oil, rapeseed oil, almond oil, palm oil, coconut oil, palm kernel oil and the like.

Any mixtures of such oil and wax components can also be used advantageously for the purposes of the present invention. It may also in some instances be advantageous to use waxes, for example cetyl palmitate, as the sole lipid component of the oil phase.

The oil phase is advantageously chosen from the group consisting of 2-ethylhexyl isostearate, octyidodecanol, isotridecyl isononanoate, isoeicosane, 2-ethylhexyl cocoate, C₁₂₋₁₅-alkyl benzoate, caprylic/capric triglyceride, dicaprylyl ether.

Particularly advantageous mixtures are those of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate.

Of the hydrocarbons, paraffin oil, squalane and squalene are to be used advantageously for the purposes of the present invention.

The oil phase can advantageously also have a content of cyclic or linear silicone oils, or consist entirely of such oils, although it is preferable to use an additional content of other oil phase components apart from the silicone oil or the silicone oils. Such silicones or silicone oils may be in the form of monomers, which are generally characterized by structural elements, as follows:

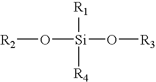

Linear silicones having two or more siloxyl units which are to be used advantageously according to the invention are generally characterized by structural elements, as follows: 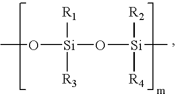
where the silicon atoms can be substituted by identical or different alkyl radicals and/or aryl radicals, which are shown here in general terms by the radicals R₁-R₄ (that is to say the number of different radicals is not necessarily limited to 4). m can assume values from 2-200 000.

Cyclic silicones to be used advantageously according to the invention are generally characterized by structural elements, as follows 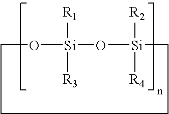
where the silicon atoms can be substituted by identical or different alkyl radicals and/or aryl radicals, which are shown here in general terms by the radicals R₁-R₄ (that is to say the number of different radicals is not necessarily limited to 4). n can assume values from 3/2 to 20. Fractions for n take into consideration that uneven numbers of siloxyl groups may be present in the cycle.

Advantageously, cyclomethicone (e.g. decamethylcyclopentasiloxane) is used as the silicone oil to be used according to the invention. However, other silicone oils are also to be used advantageously for the purpose of the present invention, for example undecamethylcyclotrisiloxane, polydimethylsiloxane, poly(methylphenylsiloxane), cetyldimethicone, behenoxydimethicone.

Also advantageous are mixtures of cyclomethicone and isotridecyl isononanoate, and those of cyclomethicone and 2-ethylhexyl isostearate.

It is, however, also advantageous to choose silicone oils of similar constitution to the above-described compounds whose organic side chains are derivatized, for example polyethoxylated and/or polypropoxylated. These include, for example, polysiloxane-polyalkyl-polyether copolymers, such as cetyl-dimethicone copolyol, (cetyl-dimethicone copolyol (and) polyglyceryl-4-isostearate (and) hexyl laurate).

Also particularly advantageous are mixtures of cyclomethicone and isotridecyl isononanoate, and of cyclomethicone and 2-ethylhexyl isostearate.

The aqueous phase of the preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol, and, in particular, one or more thickeners which can advantageously be chosen from the group consisting of silicon dioxide and aluminum silicates.

Preparations according to the invention in the form of emulsions advantageously comprise, in particular, one or more hydrocolloids. These hydrocolloids can advantageously be chosen from the group of gums, polysaccharides, cellulose derivatives, phyllosilicates, polyacrylates and/or other polymers.

Preparations according to the invention in the form of hydrogels comprise one or more hydrocolloids. These hydrocolloids can advantageously be chosen from the abovementioned group.

The gums include saps from plants or trees which harden in the air and form resins, or extracts from aquatic plants. From this group, for the purposes of the present invention, gum arabic, carob flour, tragacanth, karaya, guar gum, pectin, gellan gum, carrageen, agar, algins, chondrus, xanthan gum, for example, can be chosen advantageously.

Also advantageous is the use of derivatized gums, such as, for example, hydroxypropyl guar (Jaguar® HP 8).

The polysaccharides and polysaccharide derivatives include, for example, hyaluronic acid, chitin and chitosan, chondroitin sulfates, starch and starch derivatives.

The cellulose derivatives include, for example, methylcellulose, carboxymethylcellulose, hydroxyethylcellulose, hydroxypropylmethylcellulose.

The phyllosilicates include naturally occurring and synthetic clay earths, such as, for example, montmorillonite, bentonite, hectorite, laponite, magnesium aluminum silicates such as Veegum®. These can be used as such or in modified form, such as, for example, stearylalkonium hectorites.

In addition, silica gels can also be used advantageously.

The polyacrylates include, for example, Carbopol grades from Goodrich (Carbopol 980, 981, 1382, 5984, 2984, EDT 2001 or Pemulen TR2).

The polymers include, for example, polyacrylamides (Seppigel 305), polyvinyl alcohols, PVP, PVP/VA copolymers, polyglycols.

Preparations according to the invention in the form of emulsions comprise one or more emulsifiers. These emulsifiers can advantageously be chosen from the group of nonionic, anionic, cationic or amphoteric emulsifiers.

The nonionic emulsifiers include
a) partial fatty acid esters and fatty acid esters of polyhydric alcohols and ethoxylated derivatives thereof (e.g. glyceryl monostearates, sorbitan stearates, glyceryl stearyl citrates, sucrose stearates)
b) ethoxylated fatty alcohols and fatty acids
c) ethoxylated fatty amines, fatty acid amides, fatty acid alkanolamides
d) alkylphenol polyglycol ethers (e.g. Triton X).

The anionic emulsifiers include
a) soaps (e.g. sodium stearate)
b) fatty alcohol sulfates
c) mono-, di- and trialkylphosphoric esters and ethoxylates thereof.

The cationic emulsifiers include
a) quaternary ammonium compounds with a long-chain aliphatic radical, e.g. distearyldimonium chloride.

The amphoteric emulsifiers include
a) alkylamininoalkanecarboxylic acids
b) betaines, sulfobetaines
c) imidazoline derivatives.

In addition, there are naturally occurring emulsifiers, which include beeswax, wool wax, lecithin and sterols.

O/W emulsifiers can be advantageously chosen, for example, from the group of polyethoxylated or polypropoxylated or polyethoxylated and polypropoxylated products, e.g.:
fatty alcohol ethoxylates,
ethoxylated wool wax alcohols,
polyethylene glycol ethers of the general formula R—O—(—CH₂—CH₂—O—)ₙ—R′,
fatty acid ethoxylates of the general formula R—COO—(—CH₂—CH₂—O—)ₙ—H,
etherified fatty acid ethoxylates of the general formula R—COO—(—CH₂—CH₂—O—)ₙ—R′,
esterified fatty acid ethoxylates of the general formula R—COO—(—CH₂—CH₂—O—)ₙ—C(O)—R′,
polyethylene glycol glycerol fatty acid esters,
ethoxylated sorbitan esters,
cholesterol ethoxylates,
ethoxylated triglycerides,
alkyl ether carboxylic acids of the general formula R—O—(—CH₂—CH₂—O—)ₙ—CH₂—COOH and n are a number from 5 to 30,
polyoxyethylene sorbitol fatty acid esters,
alkyl ether sulfates of the general formula R—O—(—CH₂—CH₂—O—)ₙ—SO₃—H,
fatty alcohol propoxylates of the general formula R—O—(—CH₂—CH(CH₃)—O—)ₙ—H,
polypropylene glycol ethers of the general formula R—O—(—CH₂—CH(CH₃)—O—)ₙ—R′,
propoxylated wool wax alcohols,
etherified fatty acid propoxylates R—COO—(—CH₂—CH(CH₃)—O—)ₙ—R′,
esterified fatty acid propoxylates of the general formula R—COO—(—CH₂—CH(CH₃)—O—)ₙ—C(O)—R′,
fatty acid propoxylates of the general formula R—COO—(—CH₂—CH(CH₃)—O—)ₙ—H,
polypropylene glycol glycerol fatty acid esters,
propoxylated sorbitan esters,
cholesterol propoxylates,
propoxylated triglycerides,
alkyl ether carboxylic acids of the general formula R—O—(—CH₂—CH(CH₃)O—)ₙ—CH₂—COOH,
alkyl ether sulfates or the parent acids of these sulfates of the general formula R—O—(—CH₂—CH(CH₃)—O—)ₙ—SO₃—H,
fatty alcohol ethoxylates/propoxylates of the general formula R—O—Xₙ—Yₘ—H,
polypropylene glycol ethers of the general formula R—O—Xₙ—Yₘ—R′,
etherified fatty acid propoxylates of the general formula R—COO—Xₙ—Yₘ—R′,
fatty acid ethoxylates/propoxylates of the general formula R—COO—Xₙ—Yₘ—H.

According to the invention, particularly advantageous polyethoxylated or polypropoxylated or polyethoxylated and polypropoxylated O/W emulsifiers used are those chosen from the group of substances having HLB values of 11-18, very particularly advantageously having HLB values of 14.5-15.5, provided the O/W emulsifiers have saturated radicals R and R′. If the O/W emulsifiers have unsaturated radicals R and/or R′, or isoalkyl derivatives are present, then the preferred HLB value of such emulsifiers can also be lower or higher.

It is advantageous to choose the fatty alcohol ethoxylates from the group of ethoxylated stearyl alcohols, cetyl alcohols, cetylstearyl alcohols (cetearyl alcohols). Particular preference is given to:

polyethylene glycol(13) stearyl ether (steareth-13), polyethylene glycol(14) stearyl ether (steareth-14), polyethylene glycol(15) stearyl ether (steareth-15), polyethylene glycol(16) stearyl ether (steareth-16), polyethylene glycol(17) stearyl ether (steareth-17), polyethylene glycol(18) stearyl ether (steareth-18), polyethylene glycol(19) stearyl ether (steareth-19), polyethylene glycol(20) stearyl ether (steareth-20), polyethylene glycol(12) isostearyl ether (isosteareth-12), polyethylene glycol(13) isostearyl ether (isosteareth-13), polyethylene glycol(14) isostearyl ether (isosteareth-14), polyethylene glycol(15) isostearyl ether (isosteareth-15), polyethylene glycol(16) isostearyl ether (isosteareth-16), polyethylene glycol(17) isostearyl ether (isosteareth-17), polyethylene glycol(18) isostearyl ether (isosteareth-18), polyethylene glycol(19) isostearyl ether (isosteareth-19), polyethylene glycol(20) isostearyl ether (isosteareth-20),

polyethylene glycol(13) cetyl ether (ceteth-13), polyethylene glycol(14) cetyl ether (ceteth-14), polyethylene glycol(15) cetyl ether (ceteth-15), polyethylene glycol(16) cetyl ether (ceteth-16), polyethylene glycol(17) cetyl ether (ceteth-17), polyethylene glycol(18) cetyl ether (ceteth-18), polyethylene glycol(19) cetyl ether (ceteth-19), polyethylene glycol(20) cetyl ether (ceteth-20), polyethylene glycol(13) isocetyl ether (isoceteth-13), polyethylene glycol(14) isocetyl ether (isoceteth-14), polyethylene glycol(15) isocetyl ether (isoceteth-15), polyethylene glycol(16) isocetyl ether (isoceteth-16), polyethylene glycol(17) isocetyl ether (isoceteth-17), polyethylene glycol(18) isocetyl ether (isoceteth-18), polyethylene glycol(19) isocetyl ether (isoceteth-19), polyethylene glycol(20) isocetyl ether (isoceteth-20),

polyethylene glycol(12) oleyl ether (oleth-12), polyethylene glycol(13) oleyl ether (oleth-13), polyethylene glycol(14) oleyl ether (oleth-14), polyethylene glycol(15) oleyl ether (oleth-15),

polyethylene glycol(12) lauryl ether (laureth-12), polyethylene glycol(12) isolauryl ether (isolaureth-12),

polyethylene glycol(13) cetylstearyl ether (ceteareth-13), polyethylene glycol(14) cetylstearyl ether (ceteareth-14), polyethylene glycol(15) cetylstearyl ether (ceteareth-15), polyethylene glycol(16) cetylstearyl ether (ceteareth-16), polyethylene glycol(17) cetylstearyl ether (ceteareth-17), polyethylene glycol(18) cetylstearyl ether (ceteareth-18), polyethylene glycol(19) cetylstearyl ether (ceteareth-19), polyethylene glycol(20) cetylstearyl ether (ceteareth-20).

It is also advantageous to choose the fatty acid ethoxylates from the following group:

polyethylene glycol(20) stearate, polyethylene glycol(21) stearate, polyethylene glycol(22) stearate, polyethylene glycol(23) stearate, polyethylene glycol(24) stearate, polyethylene glycol(25) stearate,

polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate,

polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20) oleate.

The ethoxylated alkyl ether carboxylic acid or salt thereof which can be used is advantageously sodium laureth-11 carboxylate.

Sodium laureth 1-4 sulfate can be used advantageously as alkyl ether sulfate.

An advantageous ethoxylated cholesterol derivative which can be used is polyethylene glycol(30) cholesteryl ether. Polyethylene glycol(25) soyasterol has also proven successful.

Ethoxylated triglycerides which can be advantageously used are polyethylene glycol(60) Evening Primrose glycerides.

It is also advantageous to choose the polyethylene glycol glycerol fatty acid esters from the group polyethylene glycol(20) glyceryl laurate, polyethylene glycol(21) glyceryl laurate, polyethylene glycol(22) glyceryl laurate, polyethylene glycol(23) glyceryl laurate, polyethylene glycol(6) glyceryl caprate, polyethylene glycol(20) glyceryl oleate, polyethylene glycol(20) glyceryl isostearate, polyethylene glycol(18) glyceryl oleate/cocoate.

It is likewise favorable to choose the sorbitan esters from the group polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate.

Advantageous W/O emulsifiers which can be used are: fatty alcohols having 8 to 30 carbon atoms, monoglycerol esters of saturated and/or unsaturated, branched and/or unbranched alkanecarboxylic acids having a chain length of from 8 to 24, in particular 12-18, carbon atoms, diglycerol esters of saturated and/or unsaturated, branched and/or unbranched alkanecarboxylic acids having a chain length of from 8 to 24, in particular 12-18, carbon atoms, monoglycerol ethers of saturated and/or unsaturated, branched and/or unbranched alcohols having a chain length of from 8 to 24, in particular 12-18, carbon atoms, diglycerol ethers of saturated and/or unsaturated, branched and/or unbranched alcohols having a chain length of from 8 to 24, in particular 12-18, carbon atoms, propylene glycol esters of saturated and/or unsaturated, branched and/or unbranched alkanecarboxylic acids having a chain length of from 8 to 24, in particular 12-18, carbon atoms, and sorbitan esters of saturated and/or unsaturated, branched and/or unbranched alkanecarboxylic acids having a chain length of from 8 to 24, in particular 12-18, carbon atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol(2) stearyl ether (steareth-2), glyceryl monolaurate, glyceryl monocaprate, glyceryl monocaprylate.

The examples below are intended to illustrate, but not restrict, the invention. Unless stated otherwise, the numbers given are based on % by wt.

| | % by wt. |
|---|---|
| Example No. 1 | |
| O/W Cream | |
| Glyceryl stearate self-emulsifying | 4.00 |
| PEG-40 stearate | 1.00 |
| Cetyl alcohol | 3.00 |
| Caprylic/capric triglycerides | 5.00 |
| Paraffinum liquidum | 5.00 |
| Licochalcone A | 0.05 |
| Arctiin | 0.1 |
| Tocopherol | 0.1 |
| Na₃HEDTA | 0.1 |
| Preservative, perfume | q.s. |
| Polyacrylic acid | 3.00 |
| Sodium hydroxide solution 45% | q.s |
| Glycerol | 5.00 |
| Water | ad 100 |
| Example No. 2 | |
| O/W Cream | |
| Glyceryl stearate self-emulsifying | 3.00 |
| Stearic acid | 1.00 |
| Cetyl alcohol | 2.00 |
| Caprylic/capric triglycerides | 3.00 |
| Dicaprylyl ether | 4.00 |
| Paraffinum liquidum | 2.00 |
| Licochalcone A | 0.01 |
| Arctiin | 0.50 |
| Preservative, perfume | q.s. |
| Polyacrylic acid | 0.10 |
| Sodium hydroxide solution 45% | q.s. |
| Glycerol | 3.00 |
| Butylene glycol | 3.00 |
| Water | ad 100 |
| Example No. 3 | |
| O/W Cream | |
| Glyceryl stearate citrate | 2.00 |
| Stearyl alcohol | 2.00 |
| Lanolin alcohol | 1.00 |
| Caprylic/capric triglycerides | 4.00 |
| Paraffinum liquidum | 8.00 |
| Dimethicone | 1.00 |
| Licochalcone A | 0.04 |
| Arctiin | 2.00 |
| Preservative, perfume | q.s. |
| Sodium hydroxide solution 45% | q.s. |
| Glycerol | 7.50 |
| Water | ad 100 |
| Example No. 4 | |
| W/O Cream | |
| Polyglyceryl-3 diisostearate | 3.50 |
| Glycerol | 3.00 |
| Polyglyceryl-2 dipolyhydroxystearate | 3.50 |
| Licochalcone A | 0.1 |
| Arctiin | 1.00 |
| Preservative | q.s. |
| Perfume | q.s. |
| Magnesium sulfate | 0.6 |
| Isopropyl stearate | 2.0 |
| Caprylyl ether | 8.0 |
| Cetearyl isononanoate | 6.0 |
| Water | ad 100 |
| Example No. 5 | |
| W/O Emulsion | |
| Triceteareth-4 phosphate | 0.80 |
| Butylhydroxytoluene | 0.05 |
| Glyceryl lanolate | 1.70 |
| Cyclomethicone | 2.20 |
| Isopropyl palmitate | 1.00 |
| Licochalcone A | 0.10 |
| Arctiin | 1.00 |
| Polyacrylic acid | 0.50 |
| Ethylenediaminetetraacetic acid | 1.00 |
| Sodium hydroxide | q.s. |
| Citric acid | 0.01 |
| Preservative | q.s. |
| Perfume | q.s. |
| Water | ad 100 |

1.-13. (canceled)

14. An active ingredient combination comprising
(a) one or more of compounds selected from 2,3-dibenzylbutyrolactone and derivatives thereof, and
(b) licochalcone A.

15. The combination of claim 14, wherein licochalcone A is present as an aqueous extract of Radix Glycyrrhizae inflatae.

16. The combination of claim 14, wherein a ratio of component (a) component (b) is from 100:1 to 1:50.

17. The combination of claim 16, wherein the ratio is from 80:1 to 1:10.

18. The combination of claim 14, wherein component (a) comprises at least one of arctiin, arctigenin, prestegan B, matairesinol, tracheloside and trachelogenin.

19. The combination of claim 14, wherein component (a) comprises arctiin.

20. A cosmetic or dermatological preparation comprising the combination of claim 14 and a cosmetically or dermatologically acceptable carrier.

21. The preparation of claim 20, wherein the preparation comprises from 0.01% to 25% by weight of component (a), based on a total weight of the preparation.

22. The preparation of claim 21, wherein the preparation comprises from 0.1% to 20% by weight of component (a).

23. The preparation of claim 21, wherein the preparation comprises from 1% to 10% by weight of component (a).

24. The preparation of claim 20, wherein the preparation comprises from 0.0001% to 5% by weight of licochalcone A, based on a total weight of the preparation.

25. The preparation of claim 24, wherein the preparation comprises from 0.001% to 1% by weight of licochalcone A.

26. The preparation of claim 24, wherein the preparation comprises from 0.005% to 0.15% by weight of licochalcone A.

27. The preparation of claim 20, wherein the preparation further comprises
(c) one or more polyols.

28. The preparation of claim 27, wherein the preparation comprises from 0.001% to 10% by weight of component (c), based on a total weight of the preparation.

29. The preparation of claim 28, wherein the preparation comprises from 0.01% to 5% by weight of component (c).

30. The preparation of claim 29, wherein the preparation comprises from 0.05% to 2% by weight of component (c).

31. The preparation of claim 28, wherein component (c) comprises butylene glycol.

32. A cosmetic or dermatological preparation comprising (a) from 0.01% to 25% by weight of one or more of compounds selected from 2,3-dibenzylbutyrolactone and derivatives thereof and (b) from 0.0001% to 5% by weight of licochalcone A, based on a total weight of the preparation.

33. The preparation of claim 32, wherein the preparation comprises (a) from 1% to 10% by weight of one or more of compounds selected from 2,3-dibenzylbutyrolactone and derivatives thereof and (b) from 0.005% to 1% by weight of licochalcone A.

34. The preparation of claim 33, wherein the preparation comprises at least one of arctiin, arctigenin, prestegan B, matairesinol, tracheloside and trachelogenin.

35. The preparation of claim 33, wherein the preparation comprises arctiin.

36. The preparation of claim 33, wherein the preparation further comprises from 0.05% to 10% by weight of one or more polyols, based on a total weight of the preparation.

37. The preparation of claim 36, wherein the preparation comprises up to 2% by weight of the one or more polyols.

38. The preparation of claim 36, wherein the one or more polyols comprise butylene glycol.

39. A method of preventing or treating inflammatory skin conditions or for protecting skin in cases of sensitively determined and dry skin, wherein the method comprises applying the preparation of claim 20 to skin.

40. A method of preventing or treating symptoms of intrinsic or extrinsic skin aging and for preventing or treating harmful effects of ultraviolet radiation on skin, wherein the method comprises applying the preparation of claim 20 to skin.

41. A method of preventing or treating pigment disorders of skin, wherein the method comprises applying the preparation of claim 20 to skin.

42. A method of increasing ceramide biosynthesis, wherein the method comprises applying the preparation of claim 20 to skin.

43. A method of enhancing the barrier function of skin, wherein the method comprises applying the preparation of claim 20 to skin.

44. A method of preventing or treating functional disorders of skin appendages, wherein the method comprises applying the preparation of claim 20 to skin appendages.
